(19) United States
(12) Patent Application Publication
(10) Pub. No.: US 2002/0128573 A1
(43) Pub. Date: Sep. 12, 2002

(54) Therapeutic boot having heat retaining protuberances

(76) Inventor: Ronald J. Caselnova, Staten Island, NY (US)

Correspondence Address: Richard W. Goldstein, 2071 Clove Road, Staten Island, NY, 10304, US

(21) Appl. No.: 09/862,227

(22) Filed: May 22, 2001

Related U.S. Application Data
(63) Continuation of application No. 60/207,753, filed on May 30, 2000.

(51) Int. Cl. A61F 7/00; A61F 5/00

(52) U.S. Cl. 602/7; 607/96

(57) ABSTRACT

A therapeutic boot, for providing hot or cold therapy to the foot of a patient. The boot has a sole and an upper portion, whereupon a plurality of protuberances are located. The protuberances are made of or filled with a thermal substance which is capable of maintaining a heated or cooled state. Once the boot is initially heated or cooled, the foot is placed therein. When the protuberances contact the foot, heating or cooling of the contacted area is effected, and will continue for a prolonged period of time.

### CROSS REFERENCES AND RELATED SUBJECT MATTER

This application relates to subject matter contained in provisional patent application serial No. 60/207,753, filed in the United States Patent Office on May 30, 2001.

### BACKGROUND OF THE INVENTION

The invention relates to a therapeutic boot having heat retaining protuberances. More particularly, the invention relates to a non-weight bearing boot system which is capable of maintaining a heated or cooled state so as to provide thermal therapeutic care which is focused upon the area from the ankles to the toes of a patient.

Hot and cold therapy has an important role in patient rehabilitation. Cold applications are typically used to aid in decreasing inflammation due to sprains, strains, surgery, or arthritic conditions. Hot applications are most often used to increase circulation and alleviate muscle aches. Hot and cold therapy can greatly increase the quality of living for a person enduring a continuing condition such as arthritis.

Hot and cold applications are especially important for the hands and feet. The most difficult area of the body to restore circulation to is the hands and feet. In addition, the foot can be very slow to heal following surgery. Proper and precise hot and cold therapy can greatly speed recovery time for the feet.

One of the practical difficulties for hot and cold applications is keeping the heated or cooled applicator in the location where it provides the greatest therapy. It is especially difficult to maintain an applicator on the upper portion or instep of the foot. Even if the patient were to remain perfectly still, the applicator will eventually slide off the foot. In addition, virtually any application to the foot requires that the patient remain still. Thus, hot or cold applications cannot be made when the patient is at all mobile.

Currently, thermal gel-filled mitts are available to provide temperature therapy to the hand. In addition, some boots are available which are heatable to provide heat therapy to the foot.

While these units may be suitable for the particular purpose employed, or for general use, they would not be as suitable for the purposes of the present invention as disclosed hereafter.

### SUMMARY OF THE INVENTION

It is an object of the invention to produce a therapeutic boot which is capable of maintaining a cooled or heated state, so that it can be used to provide prolonged hot or cold therapy to a foot. Accordingly, the boot contains a thermal substance which has a high specific heat and thus a high thermal mass, such as a thermal gel, so that it capable of maintaining a high or low temperature for a relatively long period of time.

It is another object of the invention to provide a therapeutic boot which facilitates healing by focusing its cooling or heating therapy directly at the joints. Accordingly, the thermal substance is contained within a plurality of protuberances. The protuberances direct pressure against the foot surfaces and thereby bring the thermal application directly to the affected joints.

It is a further object of the invention to concentrate the heating or cooling effect on the area from the ankle to the toes. Accordingly, the protuberances are concentrated in the area of the boot which directly corresponds to the area from the ankle to the toes when a foot is placed therein. Since circulation is commonly poorest to the feet, the boot provides complete heating/cooling therapy.

The invention is a therapeutic boot, for providing hot or cold therapy to the foot of a patient. The boot has a sole and an upper portion, whereupon a plurality of protuberances are located. The protuberances are made of or filled with a thermal substance which is capable of maintaining a heated or cooled state. Once the boot is initially heated or cooled, the foot is placed therein. When the protuberances contact the foot, heating or cooling of the contacted area is effected, and will continue for a prolonged period of time.

To the accomplishment of the above and related objects the invention may be embodied in the form illustrated in the accompanying drawings. Attention is called to the fact, however, that the drawings are illustrative only. Variations are contemplated as being part of the invention, limited only by the scope of the claims.

### BRIEF DESCRIPTION OF THE DRAWINGS

In the drawings, like elements are depicted by like reference numerals. The drawings are briefly described as follows.

FIG. 1 is a diagrammatic perspective view of a boot according to the present invention, per se.

FIG. 2 is a diagrammatic perspective view of just the boot sole.

FIG. 3 is a cross sectional view, taken generally in the direction of line 3-3 in FIG. 1.

FIG. 4 is a diagrammatic perspective view, illustrating the boot being prepared for hot therapy using a microwave oven.

FIG. 5 is a diagrammatic perspective view, illustrating a further embodiment of just the boot sole.

### DETAILED DESCRIPTION OF THE PREFERRED EMBODIMENTS

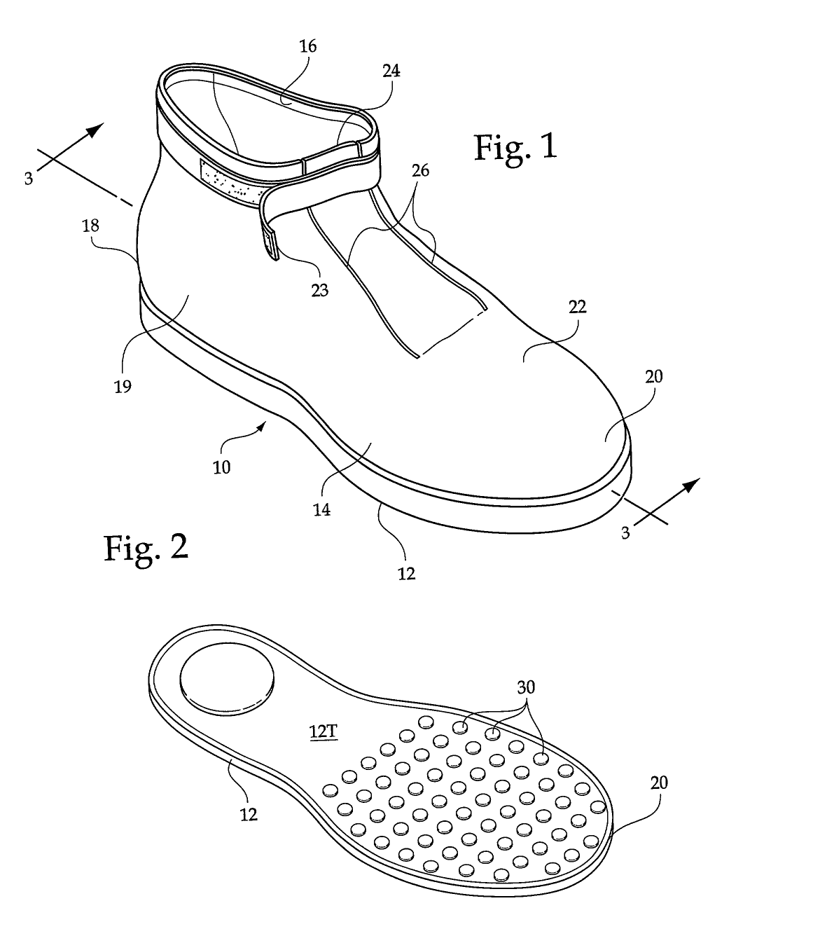

FIG. 1 illustrates a therapeutic boot 10, comprising a sole 12, and an upper portion 14. The upper portion 14 has an ankle opening 16. A tightening strap 23 is provided near the ankle opening 16 to facilitate securement onto the foot of a patient without trauma thereto. The upper portion 14, although a generally continuous surface, can be described as having a rear portion 18, side portions 19, a forward portion 20, and a roof portion 22. The boot 10 is generally configured so as to accommodate a human foot—with the toes thereof extending near the forward portion 20; the roof portion 22 covering the instep thereof; and the side portions 19 and rear portion 18 encasing the ankle thereof. The boot is configured so as to fully encase the foot to allow the patient limited motion while wearing the boot without compromising the therapeutic value thereof. However, the boot 10 is not intended to be weight-bearing, and thus is not necessarily configured to provide the same comfort and support features as ordinary footwear used for walking.

The roof portion 22 includes a tongue 24, which is formed by two longitudinal slits 26 which extend from the ankle opening 16 partially toward the forward portion 20. The longitudinal slits 26 extend substantially parallel to each other, and allow the boot 10 to adjust to accommodate different users. The tongue 24 also allows the boot 10 to be temporarily enlarged to allow insertion of an injured foot into the boot 10 without undue flexing or strain of the foot. The tightening strap 23 extends across the tongue 24 near the ankle opening 16 and allows the boot 10 to be secured onto the foot 10 after the tongue 24 has been suitably adjusted.

FIG. 2 illustrates the sole 12, which has a sole top surface 12T. In accordance with the present invention, a plurality of protuberances 30 are located on the sole 12. The protuberances 30 are spaced on the sole top surface 12T is an area extending rearwardly from the forward portion 20.

Further in accordance with the present invention, the protuberances are fabricated or filled with a thermal substance. The thermal substance has the properties of having a high specific heat, and thus a having a high thermal mass. The thermal substance can be the commonly available “blue thermal gel”, commonly used in ice packs and the like, or could be a similar substance. The thermal substance is therefore capable of being cooled or heated, and maintaining its cooled or heated state for a prolonged period of time. Thus, “thermal therapy” as used herein refers to therapy by application of either heated or cooled substances. Further, “heat retaining” as used herein refers to the ability to maintain either a heated or a cooled state. The protuberances are preferably approximately one half inch in diameter, and one half inch high.

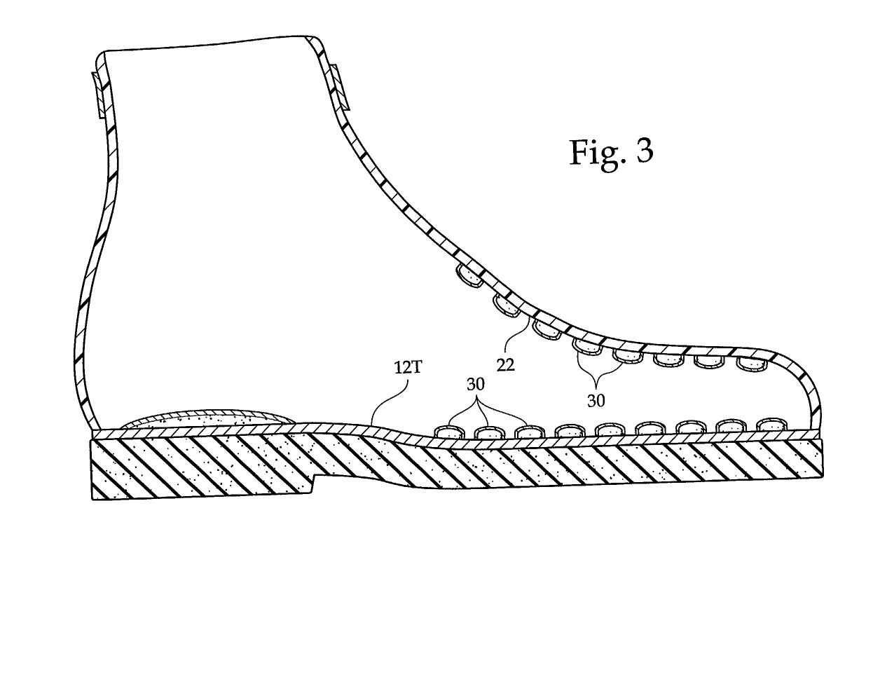

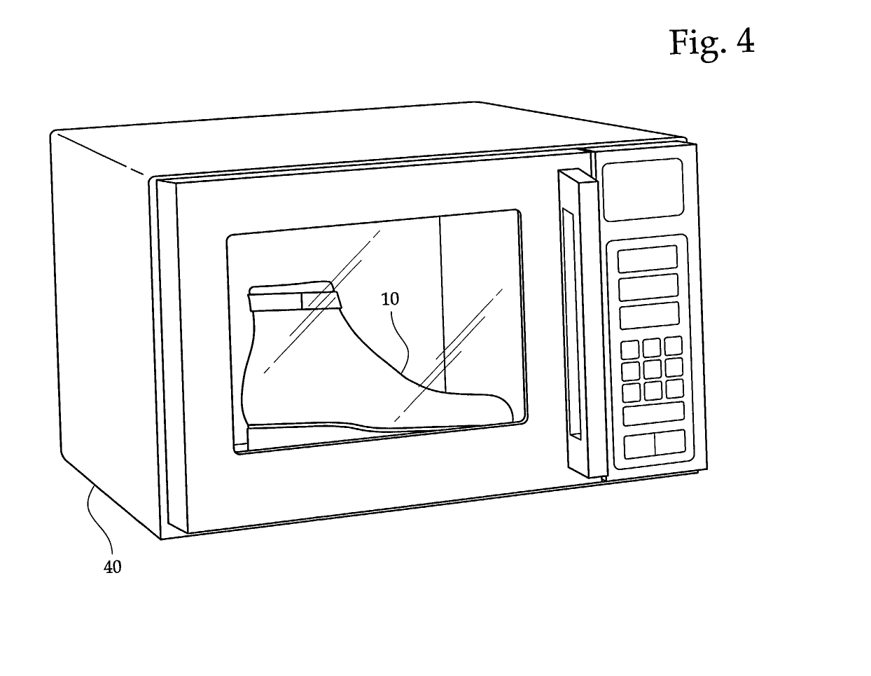

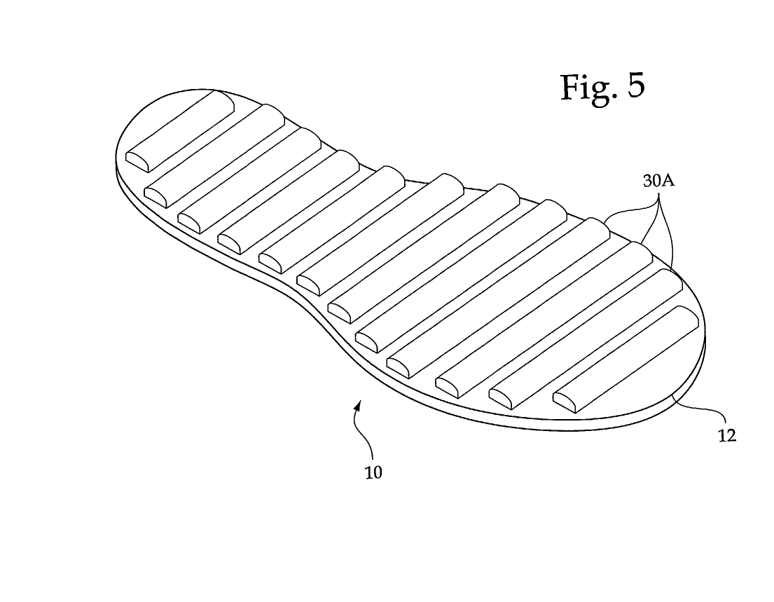

In use, the boot 10 is initially placed in a heating or cooling device 40, such as the microwave oven illustrated in FIG. 4 to effect heating of the boot 10—or a common refrigerator or freezer to effect cooling of the boot 10. The foot of the patient is then placed into the boot through the ankle opening 16. When the foot is fully inserted into the boot 10, key portions of the foot are brought into contact with the protuberances. The protuberances are preferably pliable so that they bend as the patient puts the boot on the foot. Most notably, referring to FIG. 3, protuberances 30 of the sole top surface 12T and roof portion 22 bring the protuberances 30 into contact with the toes and instep.

Additional protuberances 30 can be located to bring hot and cold therapy to the ankle, heel, or any other area of the foot deemed appropriate. Accordingly, the protuberances 30 are preferably distributed throughout the interior of the boot 10, including on the underside of the tongue 24, to effect heating or cooling of the entire foot. FIG. 5 illustrates a further embodiment of the sole 12 of the boot 10, wherein a series of transverse protuberances 30A are employed. In this embodiment, the protuberances are bar-like projections which extend across the boot, and thus across the foot. Further experimentation will determine those configurations and patterns which have the greatest therapeutic value. Such configurations can include mixing round and bar-like protuberances within the same boot.

Once the foot is in place, the protuberances will provide the necessary hot or cold therapy to the foot for a prolonged period of time. The length of time for which the boot is effective is limited only by the properties and quantity of the thermal substance used. The precise substance which is best suited for meeting these goals may be determined by empirical experimentation. Accordingly, the present invention is not limited to any specific thermal substance.

It should also be noted that the principles of the boot according to the present invention can apply equally well to a “mitt” or glove. That is, lining the interior surfaces of a mitt with the protuberances which have heat retaining characteristics would allow hot or cold therapy to be imparted to the hands with similar benefits as can be achieved by the boot of the present invention which employs such protuberances.

In conclusion, herein is presented a therapeutic boot which brings hot or cold therapy directly to the areas of the foot where it is most needed through the use of a plurality of protuberances made or filled with a thermal substance. The use of these protuberances increases the effectiveness of the hot or cold therapy, maintains contact with the foot despite movement by the patient, and prolongs the period of effective therapy. The invention as described may be embodied in the form illustrated in the example provided in the drawing figures. Numerous variations are still possible while adhering to the spirit of the invention. Such variations are contemplated as being a part of the present inventive concept.

What is claimed is:

1. A therapeutic boot, for selectively providing hot and cold therapy to the foot of a patient, comprising:
a sole and an upper portion, the sole having a sole top surface, the upper portion having a forward and a rear portion, an ankle opening is located near the rear portion, the upper portion defining an interior which is configured so as to accommodate a human foot with the toes thereof extending near the forward portion;
a thermal substances located on the sole top surface, the thermal substance capable of selectively being cooled and heated and maintaining one of a cooled state and a heated state for a prolonged period so as to provide thermal therapy to the foot.

2. The therapeutic boot as recited in claim 1, further comprising a plurality of protuberances located on the sole, the protuberances having the thermal substance so as to directly impart thermal therapy to the foot therefrom.

3. The therapeutic boot as recited in claim 2, wherein the upper portion includes a roof portion having a pair of longitudinal slits which extend from the ankle opening toward the forward portion, the longitudinal slits defining a tongue which allows the boot to be temporarily enlarge to allow insertion of the foot without undue flexing of the foot.

4. The therapeutic boot as recited in claim 3, further comprising a tightening strap, extending across the tongue near the ankle opening for allowing the boot to be secured on the foot after it has been adjusted to said foot.

4. The therapeutic boot as recited in claim 4, wherein the protuberances are distributed on the roof of the upper portion.

5. The therapeutic boot as recited in claim 4, wherein at least some of the protuberances are substantially one-half inch high.

6. The therapeutic boot as recited in claim 2, wherein at least some of the protuberances are transverse protuberances which are bar-like projections which extend across the boot.
